(19) United States
(12) Patent Application Publication
(10) Pub. No.: US 2009/0264988 A1
(43) Pub. Date: Oct. 22, 2009

(54) Stent Graft Delivery System Including Support for Fenestration in Situ and a Mechanism for Modeling

(75) Inventors: Masoumeh MAFI, Santa Rosa, CA (US); Walter BRUSZEWSKI, Guerneville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/105,548

(22) Filed: Apr. 18, 2008

(51) Int. Cl. A61F 2/06

(52) U.S. Cl. 623/1.23; 623/1.11; 623/1.13; 623/1.2

(57) ABSTRACT

A system and method for delivering a self-expanding stent graft within a segment of a body vessel having a branch vessel extending therefrom. The graft includes one or more self-expanding stents for anchoring the graft to the vessel wall and has a stent-free body portion positionable across the branch vessel. The graft delivery system includes an expandable fenestration support structure at the distal end thereof that is positioned within the graft during delivery. Once the graft has been delivered and expanded into apposition with the vessel wall, the support structure may be expanded therein to press the unsupported body portion of the graft against the branch vessel such that a separate puncture device may be delivered to create a fenestration in the graft for perfusion of the branch vessel. In addition, the expanded fenestration support structure reduces any wrinkles in the graft without a secondary procedure.

### FIELD OF THE INVENTION

The present invention relates generally to methods and systems for delivering a graft through a body lumen for the treatment of vascular disease.

### BACKGROUND OF THE INVENTION

Prostheses for implantation in blood vessels or other similar organs of the living body are, in general, well known in the medical art. For example, prosthetic vascular grafts constructed of biocompatible materials, such as Dacron or expanded, porous polytetrafluoroethylene (PTFE) tubing, have been employed to replace or bypass damaged or occluded natural blood vessels. In general, endovascular grafts typically include a graft anchoring component that operates to hold the tubular graft in its intended position within the blood vessel. Most commonly, the graft anchoring component is one or more radially compressible stents that are radially expanded in situ to anchor the tubular graft to the wall of a blood vessel or anatomical conduit. Thus, endovascular grafts are typically held in place by mechanical engagement and friction due to the opposition forces provided by the expandable stents.

In general, rather than performing an open surgical procedure to implant a bypass graft that may be traumatic and invasive, stent grafts are preferably deployed through a less invasive intraluminal delivery. More particularly, a lumen or vasculature is accessed percutaneously at a convenient and less traumatic entry point, and the stent graft is routed through the vasculature to the site where the prosthesis is to be deployed. Intraluminal deployment is typically effected using a delivery catheter with coaxial inner and outer tubes arranged for relative axial movement. For example, a self-expanding stent graft may be compressed and disposed within the distal end of an outer catheter tube distal of a stop fixed to the inner member. The catheter is then maneuvered, typically routed though a body lumen until the end of the catheter and the stent graft is positioned at the intended treatment site. The stop on the inner member is then held stationary while the outer tube of the delivery catheter is withdrawn. The inner member prevents the stent graft from being withdrawn with the sheath. As the sheath is withdrawn, the stent graft is released from the confines of the sheath and radially self-expands so that at least a portion of it contacts and substantially conforms to a portion of the surrounding interior of the lumen, e.g., the blood vessel wall or anatomical conduit.

Grafting procedures are also known for treating aneurysms. Aneurysms result from weak, thinned blood vessel walls that “balloon” or expand due to aging, disease and/or blood pressure in the vessel. Consequently, aneurysmal vessels have a potential to rupture, causing internal bleeding and potentially life threatening conditions. Grafts are often used to isolate aneurysms or other blood vessel abnormalities from normal blood pressure, reducing pressure on the weakened vessel wall and reducing the chance of vessel rupture. As such, a tubular endovascular graft may be placed within the aneurysmal blood vessel to create a new flow path and an artificial flow conduit through the aneurysm, thereby reducing if not nearly eliminating the exertion of blood pressure on the aneurysm.

While aneurysms can occur in any blood vessel, most occur in the aorta and peripheral arteries. Depending on the region of the aorta involved, the aneurysm may extend into areas of bifurcation or segments of the aorta from which smaller “branch” arteries extend. Various types of aortic aneurysms may be classified on the basis of the region of aneurysmic involvement. For example, thoracic aortic aneurysms include aneurysms present in the ascending thoracic aorta, the aortic arch, and branch arteries that emanate therefrom, such as subclavian arteries. Thoracoabdominal aortic aneurysm include aneurysms present in the descending thoracic aorta and branch arteries that emanate therefrom, such as thoracic intercostal arteries and/or the suprarenal abdominal aorta and branch arteries that emanate therefrom, such as renal, superior mesenteric, celiac and/or intercostal arteries. Lastly, abdominal aortic aneurysms include aneurysms present in the pararenal aorta and the branch arteries that emanate therefrom, such as the renal arteries.

Unfortunately, not all patients diagnosed with aortic aneurysms are presently considered candidates for endovascular grafting. This is largely due to the fact that most of the endovascular grafting systems of the prior art are not designed for use in regions of the aorta from which side branches extend. The deployment of endovascular grafts within regions of the aorta from which branch arteries extend present additional technical challenges because, in those cases, the endovascular graft must be designed, implanted and maintained in a manner which does not impair the flow of blood into the branch arteries.

In order to accommodate side branches, a stent graft having a fenestration or opening in a side wall thereof is utilized. The fenestration is positioned to align with the ostium of the branch vessel after deployment of the stent graft. In use, the proximal end of the graft having one or more side openings is securely anchored in place, and the fenestrations or openings are configured and deployed to avoid blocking or restricting blood flow into the side branches. In some cases, another stent graft, often referred to as a branch graft, may then be deployed through the fenestration into the branch vessel to provide a path for blood flow to the branch vessel. One issue that exists in such a procedure is how to accurately position a fenestration creating element in relation to the branch vessel. If the position of a fenestration is offset with respect to a branch vessel when the stent graft is deployed, it may be difficult to deploy guidewires and catheters from the stent graft into the branch vessel to enable correct positioning of the branch vessel stent graft, which in turn may result in the branch graft being deployed in such a manner that it kinks to such an extent that blood flow will not occur therethrough. Thus, there remains a need in the art for the development of new endovascular grafting systems and methods for providing perfusion to side branch vessels.

### SUMMARY OF THE INVENTION

A system and method in accordance with an embodiment hereof includes a graft delivery system for delivering a stent graft within a segment of a body vessel having a branch vessel extending therefrom. The graft includes an intermediate, unsupported or stent-free body portion positionable across the branch vessel with one or more self-expanding stents provided at a proximal and/or distal end thereof for anchoring the graft to a vessel wall. The delivery system includes an expandable fenestration support structure at the distal end thereof that is positioned within the graft during delivery. Once the graft has been delivered and expanded into apposition with the vessel wall, the fenestration support structure may be expanded therein to press the otherwise unsupported body portion of the graft against the branch vessel, such that a separate puncture device may be delivered to create a fenestration in the side of the graft for perfusion of the branch vessel. The unsupported body portion of the graft is thus temporarily held in place by the expanded fenestration support structure until the fenestration is created. Thus, the expanded fenestration support structure of the graft delivery system facilitates fenestration in situ by providing radial support to the graft for branch fenestration operations. In addition, the expanded fenestration support structure models or reduces the wrinkles of the graft without a secondary procedure.

### BRIEF DESCRIPTION OF DRAWINGS

The foregoing and other features and advantages will be apparent from the following description as illustrated in the accompanying drawings. The accompanying drawings, which are incorporated herein and form a part of the specification, further serve to explain the principles of embodiments according to the present invention. The drawings are not to scale.

FIG. 1 is a schematic side view of an embodiment of a graft delivery system having a fenestration support system at a distal portion thereof.

FIG. 2 is a cross-sectional view of the graft delivery system taken along line A-A of FIG. 1.

FIG. 3 is an illustration of an enlarged side view of the distal portion of the graft delivery system of FIG. 1, wherein the fenestration support system is in an unexpanded configuration.

FIG. 4 is an illustration of an enlarged side view of the distal portion of the graft delivery system of FIG. 1, wherein the fenestration support system is in an expanded configuration.

FIG. 5 is a close up view of a portion of an endovascular graft to be deployed by the graft delivery system of FIG. 1.

FIG. 6 is an enlarged side view of the distal portion of the graft delivery system of FIG. 1 having an expanded endovascular graft pictured thereon, wherein the fenestration support system is in the expanded configuration.

FIG. 7 is an illustration of a distal portion of an expanded graft partially expanded with the tip still captured to a graft delivery system.

FIGS. 8-11 illustrate a method of creating a fenestration in a graft in situ according to an embodiment hereof.

### DETAILED DESCRIPTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. For the graft the proximal end refers to the end of the graft material nearest the heart by way of blood flow path, while distal is the end farthest from the heart by way of blood flow path.

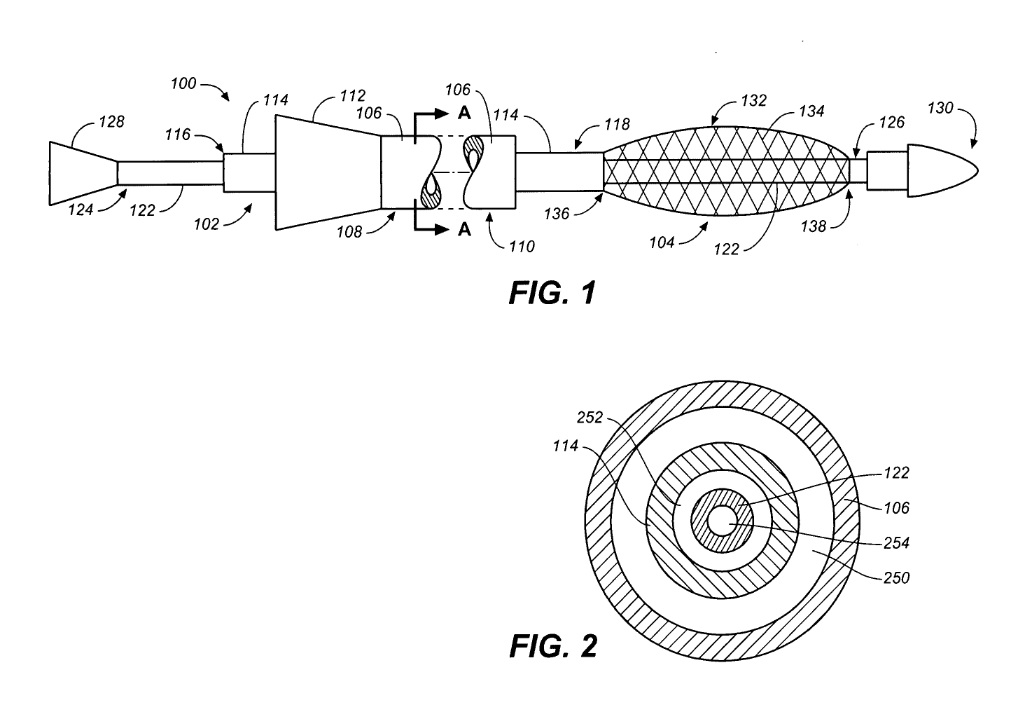

The following detailed description is merely exemplary in nature. Although the description herein is in the context of treatment of blood vessels such as the coronary, carotid and renal arteries, embodiments according to the present invention may also be used in any other body passageways where it is deemed useful. Further details and description of embodiments are provided below with reference to FIGS. 1-11.

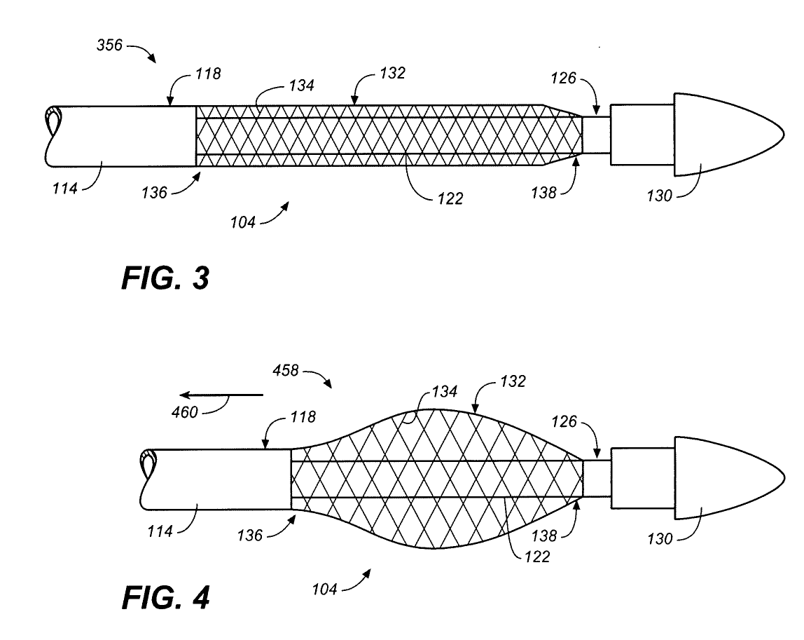

FIGS. 1-2 illustrate a graft delivery system 100 having a proximal portion 102 and a distal portion 104. FIG. 1 is a schematic side view of system 100, while FIG. 2 is a cross-sectional view taken along line A-A of FIG. 1. Graft delivery system 100 includes a retractable sheath 106 having a proximal end 108 and a distal end 110, an intermediate shaft 114 having a proximal end 116 and a distal end 118, and an inner shaft 122 having a proximal end 124 and a distal end 126. Retractable sheath 106 is provided to cover an endoluminal prosthesis (not shown in FIG. 1) mounted on the distal portion 104 of system 100 when system 100 is tracked through a body lumen to the deployment site. As illustrated in FIG. 1, retractable sheath 106 is shown in a retracted position. Intermediate shaft 114 extends through retractable sheath 106, and inner shaft 122 extends through intermediate shaft 114 to a distal tip 130 of system 100. Distal tip 130 is coupled to distal end 126 of inner shaft 122, and may be tapered and flexible to provide trackability through the vasculature. In addition, delivery system 100 may include a radiopaque marker (not shown) allowing for accurate positioning of the delivery system prior to deployment of the stent-graft.

As shown in FIG. 2, retractable sheath 106 defines a lumen 250 extending there through. Intermediate shaft 114 extends through lumen 250 of retractable sheath 106. Intermediate shaft 114 defines a lumen 252 extending there through. Inner shaft 122 extends through lumen 252 of intermediate sheath 114. Inner shaft 122 may define a guidewire lumen 254 for receiving a guidewire (not shown) there through. Inner shaft 122 may be advanced over an indwelling guidewire to track system 100 to the target site. Alternatively, inner shaft 122 may instead be a solid rod (not shown) without a lumen extending there through. In an embodiment where inner shaft 122 is a solid rod, system 100 may be tracked to the target site with the assistance of tapered distal tip 130.

In an un-retracted, distally extended position, retractable sheath 106 restrains a self-expanding graft in a constrained diameter or delivery configuration within distal end 110 thereof. Retractable sheath 106 extends to proximal portion 102 of system 100 and is movable in an axial direction along and relative to intermediate shaft 114 via an actuator, such as a handle 112, to selectively release the graft located about distal portion 104 of system 100. Handle 112 may be a push-pull actuator that is attached or connected to proximal end 108 of retractable sheath 106. In order to allow expansion of the graft, handle 112 is pulled proximally relative to intermediate shaft 114 to retract sheath 106. Alternatively, the actuator may be a rotatable knob (not shown) that is attached or connected to proximal end 108 of retractable sheath 106, such that when the knob is rotated retractable sheath 106 is retracted in a proximal direction to allow expansion of the graft. Thus, when the actuator is operated, i.e., manually turned or pulled, retractable sheath 106 is proximally retracted relative to intermediate shaft 114.

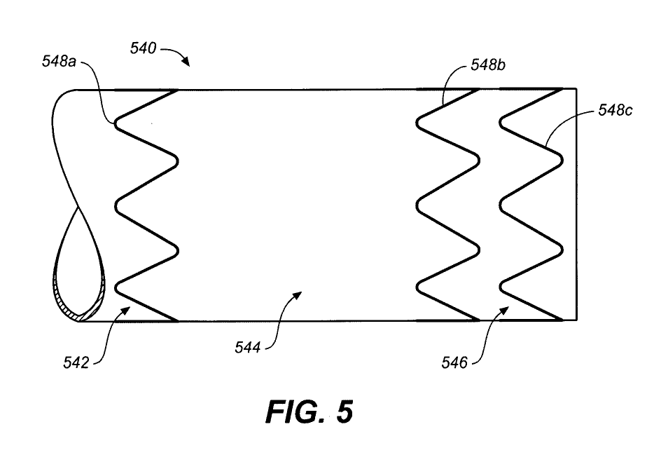

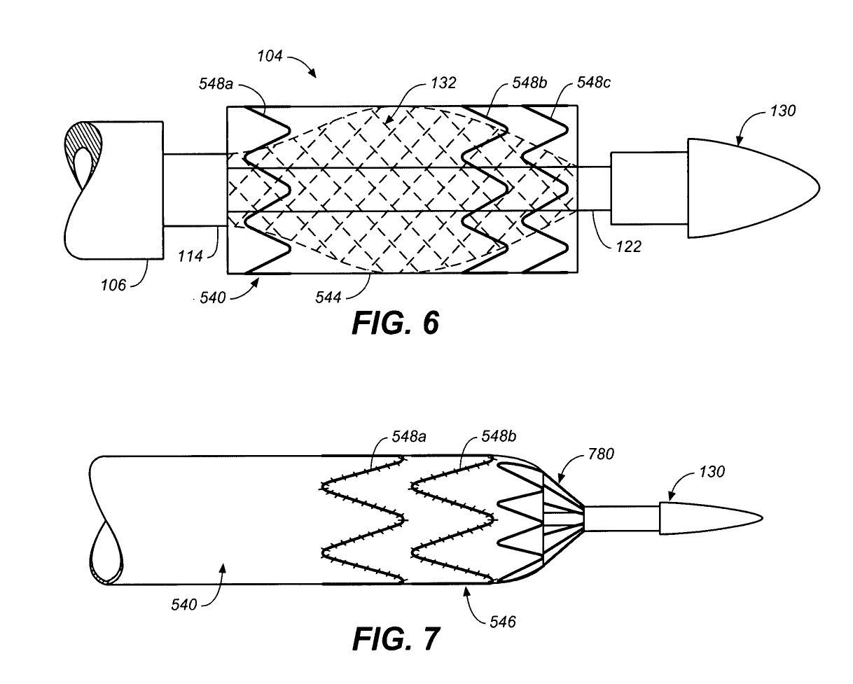

Inner shaft 122 is also movable in an axial direction along and relative to intermediate shaft 114 and extends to proximal portion 102 of system 100 where it may be controlled via a handle 128 to selectively expand an expandable fenestration support structure 132. Expandable fenestration support structure 132 is located at distal portion 104 of system 100, and includes a tubular braided structure or mesh 134. As explained in more detail herein with reference to FIGS. 3-4, fenestration support structure 132 cooperates to provide an expansion framework that is controlled to be movable between a reduced-diameter delivery configuration and an enlarged-diameter expanded configuration.

A proximal end 136 of fenestration support structure 132 is attached to distal end 118 of intermediate shaft 114, and a distal end 138 of fenestration support structure 132 is attached to distal end 126 of inner shaft 122. While holding proximal end 116 of intermediate shaft 114 fixed, inner shaft 122 may be proximally retracted via handle 128 within intermediate shaft 114. When inner shaft 122 is proximally retracted, the attachment point between fenestration support structure 132 and intermediate shaft 114 remains fixed such that fenestration support structure 132 radially expands. Fenestration support structure 132 may be attached to intermediate shaft 114 and inner shaft 122 in any suitable manner known in the art. For example, the connection may be formed by welding, such as by resistance welding, friction welding, laser welding or another form of welding such that no additional materials are used to connect fenestration support structure 132 to shafts 114, 122. Alternatively, fenestration support structure 132 can be connected to shafts 114, 122 by soldering, by the use of an adhesive, by the addition of a connecting element there between, or by another mechanical method.

Similar to handle 112 explained above, handle 128 may be a push-pull actuator that is attached or connected to proximal end 124 of inner shaft 122 to expand fenestration support structure 132 such that when handle 128 is pulled while holding proximal end 116 of intermediate shaft 114 fixed, inner shaft 122 is retracted in a proximal direction to expand fenestration support structure 132. Similarly, when handle 128 is pushed while holding proximal end 116 of intermediate shaft 114 fixed, inner shaft 122 is advanced in a distal direction to elongate or unexpand fenestration support structure 132 to allow for removal. Alternatively, the actuator may be a rotatable knob (not shown) that is attached or connected to proximal end 124 of inner shaft 122 such that when the knob is rotated, inner shaft 122 operates to expand or elongate fenestration support structure 132. Thus, when the actuator is operated, i.e., manually turned or pulled, inner shaft 122 is proximally retracted within intermediate shaft 114. Although embodiments are described with inner shaft 122 being movable relative to intermediate shaft 114 to expand fenestration support structure 132, it should be apparent to one of ordinary skill in the art that fenestration support structure 132 is expanded by shortening the distance between ends 136, 138 thereof. Thus, in another embodiment, fenestration support structure 132 may be expanded by distally advancing intermediate shaft 114 while holding inner shaft 122 stationary.

Referring now to FIGS. 3-4, fenestration support structure 132 is movable from an unexpanded configuration 356 shown in FIG. 3 to an expanded configuration 458 shown in FIG. 4. In unexpanded configuration 356, fenestration support structure 132 is relatively straight cylindrical or tubular structure with a minimized delivery profile such that graft delivery system 100 may be advanced to the target site. Fenestration support structure 132 is then radially expanded via proximal retraction of inner shaft 122 as indicated by directional arrow 460 to expanded configuration 458 shown in FIG. 4. In expanded configuration 458, fenestration support structure 132 assumes a spherical shape. Spherical as used herein is intended to include ellipsoidal and/or cylindrical shapes. Fenestration support structure 132 is expanded to the spherical shape in situ within the graft to press an unsupported stent-free body portion of the graft against a vessel wall, such that a separate puncture device may be delivered to create a fenestration in the side of the graft for perfusion of a side branch vessel. During the fenestration procedure, open spaces in tubular braided structure or mesh 134 allow blood or other fluid to flow there through, such that the blood vessel is not blocked or occluded. Once the fenestration has been created in the side wall of the graft, inner shaft 122 may be advanced in a distal direction to collapse fenestration support structure 132 back to unexpanded configuration 356. Once fenestration support structure 132 is radially collapsed, graft delivery system 100 may be retracted and withdrawn.

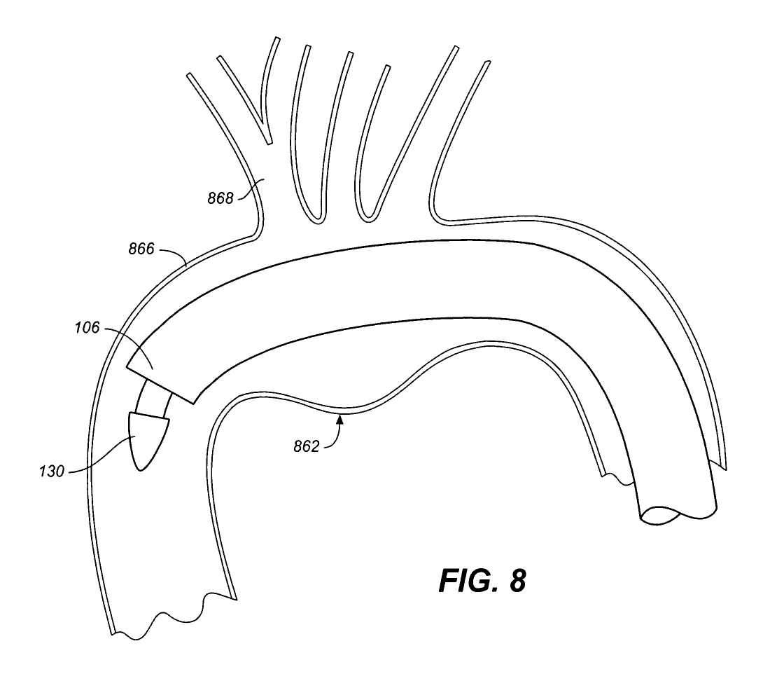

FIG. 5 is a side view of a portion of an endovascular graft 540 to be deployed by graft delivery system 100. Graft 540 is a tubular synthetic graft having a proximal portion 546, an intermediate body portion 544, and a distal portion 542. A current example of such a device is a Valiant Thoracic Stent Graft sold by Medtronic in Europe since 2005, with a modified configuration of stent distributions may be used. Proximal and distal portions 546, 542, respectively, include graft material having one or more radially compressible annular support members or stents attached thereto. FIG. 5 illustrates three stents 548a, 548b, 548c attached to graft 540; however, a greater or lesser number of stents may be utilized. Stents 548a, 548b, 548c may be self-expanding cylindrical rings that bias the proximal and distal ends of graft 540 into apposition with an interior wall of a body lumen (not shown). Stents 548a, 548b, 548c may be attached or mechanically coupled to the graft material by various means, such as, for example, by stitching or suturing onto either the inside or outside of graft 540. Intermediate body portion 544 is solely graft material having no radial support along its length, i.e., is stent-free and unsupported, and extends between proximal and distal supported graft material portions 546, 542. As such, body portion 544 is relatively flexible permitting placement of the prosthesis in a highly curved anatomy and reducing stresses on graft 540. The length of the unsupported body portion 544 may vary and in one embodiment is approximately 1 to 2 cm.

As shown in FIG. 5, radially compressible stents 548a, 548b, 548c may be attached to both the proximal and distal portions 546, 542 of graft 540.

Stents for use herein are preferably self-expanding spring members that are deployed by release from a restraining mechanism such as retractable sheath 106. For example, the stents may be constructed of a superelastic material such as nitinol. The stents may have any suitable configuration. For example, the stents may be wavelike or sinusoidal patterned wire rings, a series of connected compressible diamond structures or other compressible spring members biased in a radially outward direction, which when released, bias the prosthesis into conforming fixed engagement with an interior surface of the vessel. Examples of such annular support members are described, for example, in U.S. Pat. No. 5,713,917 and U.S. Pat. No. 5,824,041, which are incorporated by reference herein in their entirety. When used in an aneurysm exclusion device, the stents have sufficient radial spring force and flexibility to conformingly engage the prosthesis with the body lumen inner wall, to avoid excessive leakage, and prevent pressurization of the aneurysm, i.e., to provide a leak-resistant seal. Although some leakage of blood or other body fluid may occur into the aneurysm isolated by the graft prosthesis, an optimal seal will reduce the chances of aneurysm pressurization and resulting rupture.

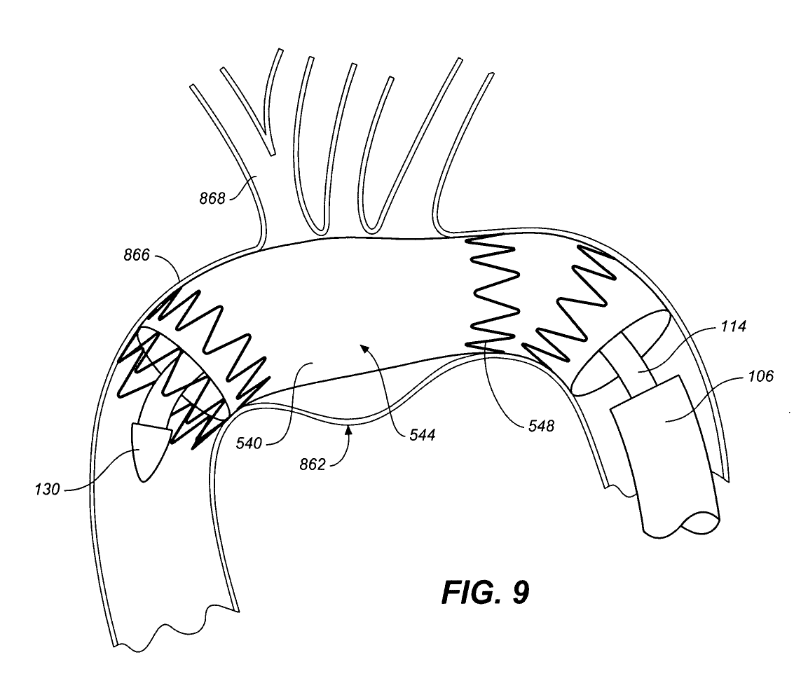

FIG. 6 is a side view of distal portion 104 of graft delivery system 100 having an expanded endovascular graft 540 pictured thereon. Graft 540 is mounted on distal portion 104 of graft delivery system 100 such that the intermediate unsupported body portion 544 of graft 540 is located over fenestration support structure 132. Once expanded, fenestration support structure 132 operates to press unsupported body portion 544 of graft 540 against a vessel wall such that a separate puncture device may be delivered to create a fenestration in the side of graft 540 for perfusion of a side branch vessel. In addition, expanded fenestration support structure 132 models or reduces the wrinkles of graft 130, thus avoiding a secondary procedure to do so.

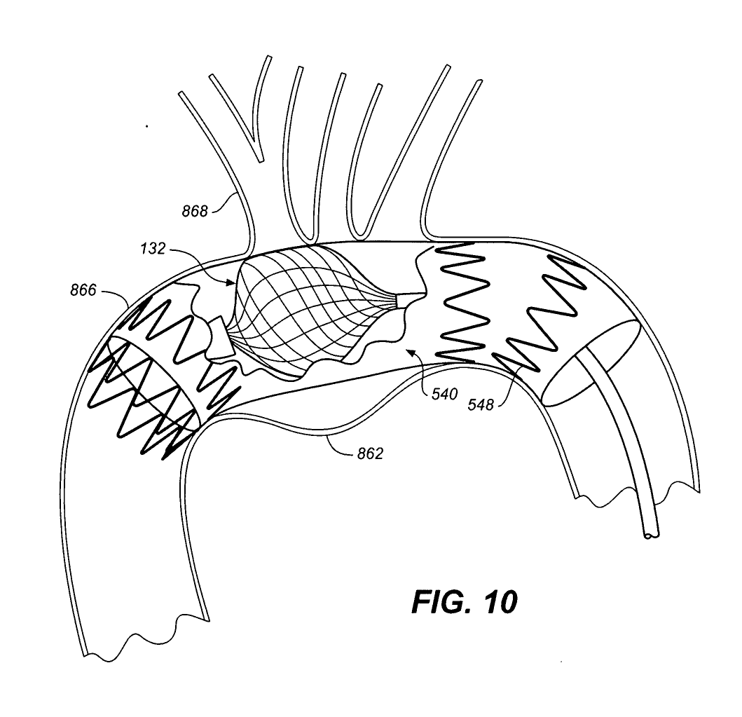

Similarly, FIG. 7 is an illustration of a distal portion of a graft partially expanded with the tip still captured to a graft delivery system. FIG. 7 shows attachment struts 780 attached to proximal portion 546 of graft 540 and extending to distal end 126 of inner shaft 122 for acting as a means for retaining graft 540 in place during delivery. Attachment struts 780 when released self-expand to their full diameter. Other means may be used for retaining graft 540 in place within graft delivery system 100 during delivery. For example, in addition to or in the alternative, graft 130 may be held in frictional engagement with graft delivery system 100 by the inclusion of slots, ridges, pockets, or other prosthesis retaining features (not shown) formed into the exterior surface of intermediate shaft 114 to further ensure secure mounting of graft 130 as it is tracked transluminally to the target site. In addition, a cap (not shown) may be coupled to distal end 126 of inner shaft 122 to retain the graft 540 in a radially compressed configuration. An actuator at the proximal portion of the system may precisely control the release of the graft 540 from the cap and from the radially compressed configuration. A more extensive description of this mechanism can be had by referring to U.S. Pat. No. 7,264,632 to Wright and U.S. patent application Ser. No. 12/052,989 to Glynn et al. filed on 21 Mar. 8, both incorporated in their entirety herein by reference.

Retractable sheath 106, intermediate shaft 114, and inner shaft 122 may be constructed of any suitable flexible polymeric material. Non-exhaustive examples of material for the catheter shafts are polyethylene terephalate (PET), nylon, polyethylene, PEBAX, or combinations thereof, either blended or co-extruded. Optionally, a portion of the catheter shafts may be constructed as a composite having a reinforcement material incorporated within a polymeric body to enhance strength, flexibility, and/or toughness. Suitable reinforcement layers include braiding, wire mesh layers, embedded axial wires, embedded helical or circumferential wires, and the like. In an embodiment, the proximal portions of the catheter shafts may in some instances be constructed from a reinforced polymeric tube, for example, as shown and described in U.S. Pat. No. 5,827,242 to Follmer et al. which is incorporated by reference herein in its entirety. The catheter shafts may have any suitable working length, for example, 550 mm-600 mm, to extend to a target location where a graft is to be implanted.

Fenestration support structure 132 has sufficient mechanical strength to press at least a portion of the graft to a vessel wall of a body lumen. In another embodiment, fenestration support structure 132 may be constructed from a tubular braided structure including a plurality of metallic wires or polymeric filaments woven together to form a tubular structure. Non-exhaustive examples of metallic materials for fenestration support structure 132 are stainless steel, cobalt based alloys (605L, MP35N), titanium, tantalum, ceramic, and superelastic nickel-titanium alloy, such as nitinol. Non-exhaustive examples of polymeric materials for fenestration support structure 132 are polyurethane, polyethylene terephalate (PET), nylon, polyethylene, PEBAX, or combinations thereof, either blended or co-extruded.

The fenestration support structure can be used with electrically conductive or high temperature graft puncture devices whose use is described below. In instances where an electrically conductive puncture device such as RF or plasma utilizing wires or electrodes as are used to create a localized area or volume of graft material vaporizing energy, or resistive heating elements which provide a localized melt cutting of the graft material, the wires and or other elements of the fenestration support structure can be coated to prevent grounding or errant conduction of electricity or electric fields or currents away from the wire or electrodes. Such coatings may be non-conductive ceramic, polyimide Kapton, hi-temp Parylene, or other heat resistant dielectric material which can form a coating. Coating thicknesses of approximately 0.001 inches have been found to be sufficient. A source for useful parylene coatings is Specialty Coating Systems, of Indianapolis, Ind.

Graft 540 is a tubular synthetic graft constructed from a suitable biocompatible material such as DACRON or other polyester fabric, or PTFE (polytetrafluoroethylene). The graft material is thin-walled so that graft 540 may be compressed into a small diameter, yet is capable of acting as a strong, leak-resistant fluid conduit when expanded to a cylindrical tubular form. In one embodiment, unsupported intermediate portion 544 of graft 540 may include a printed pattern of radiopaque markings to delineate the surface of the graft cloth radiographically as described in U.S. patent application Ser. No. ______, filed ______ (Atty Docket No, P30242), which is herein incorporated by reference in its entirety. Such radiopaque markings will assist in creating the fenestration in the graft to allow blood flow into the side branch vessels.

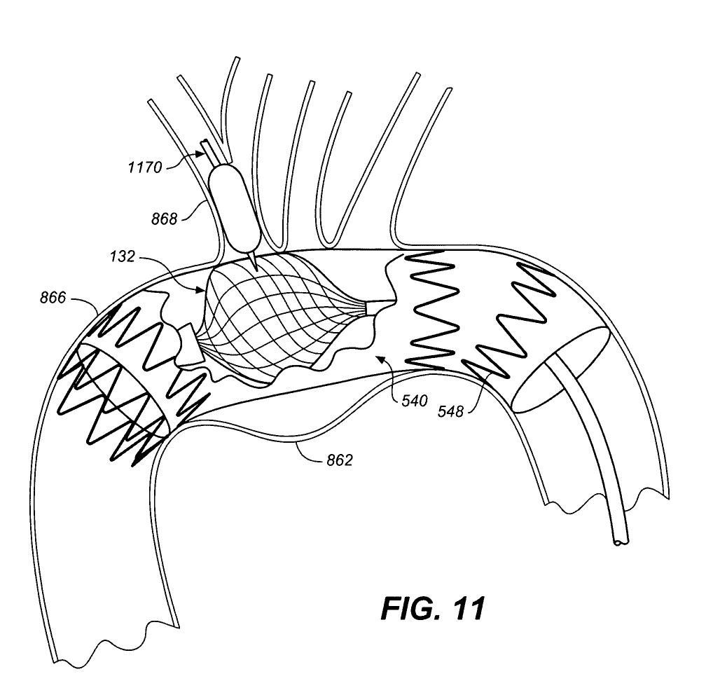

Referring now to FIGS. 8-11, a method of implanting a graft within an aneurysm 862 and creating a fenestration in a side wall of a graft in situ using a graft delivery system according to an embodiment hereof is described. FIG. 8 is a side view of graft delivery system 100 disposed within aortic arch 866. Aortic arch 866 has multiple side branch vessels 868 extending therefrom, including the left subclavian artery, the left common carotid artery, and the brachlocephalic artery, which further branches into the right subclavian artery and the right common carotid artery. The following method of creating a fenestration in a side wall of a graft in situ is described to provide perfusion to the brachlocephalic artery, but it will be understood that the method may be utilized for providing perfusion to the left subclavian artery or the left common carotid artery, as well as branch side vessels of other vessels when the system hereof is used in a vessel other than the aortic arch. The graft delivery system is tracked to and properly positioned within aortic arch 866 such that the graft to be delivered spans aneurysm 862 and initially covers side branch vessels 868. In use, the self-expanding graft 540 is preloaded into the delivery system with the stents 548a, 548b, 548c held in a radially compressed configuration. Retractable sheath 106 is placed over the graft to restrain the graft in the compressed configuration and prevent it from damaging or catching on the luminal wall as it is delivered to the aneurysm site. The graft is delivered in a compressed state via retractable sheath 106. Methods and apparatus for delivering the graft intravascularly are generally known in the art and may be used to place the graft delivery system within the vasculature and deliver the graft to the deployment site. For example, the graft may be guided to the deployment site using fluoroscopic imaging.

When a distal portion of the graft delivery system is located at the deployment site, retractable sheath 106 is proximally retracted to allow the graft to self-expand into apposition with the vessel wall. As shown in FIG. 9, graft 540 is in its deployed or expanded configuration. Stents 548a, 548b, 548c are biased in a radially outward direction, such that graft 540 is anchored within the vessel to thereby provide an artificial lumen for the flow of blood. Graft 540 includes an intermediate unsupported or stent-free body portion 544 that extends across the branch vessels 868. The aneurysm 862 shown in FIG. 9 is idealized for illustration purposes, as being directly opposite the fenestrable branches 868. The actual location of aneurysm and their size in the aortic arch, while not likely to involve the ostia of the branches which tend to be strong a durable tubular structure, can generally randomly occur in other parts of the ascending, arching and descending portion of the aorta. Thus while a small laterally unsupported fenestration support structure is shown having a general shape and volume which appears proportionally sized to the adjacent aneurysm, in practice a fenestration support structure would be sized to be a longer curving cylinder shaped which spans between healthy tissue areas surrounding the aneurysm would be chosen to so that the support in the area where branch fenestration is needed would be sufficient. In many instances the actual rotation and longitudinal configuration of the aneurysm with respect to the branch vessel openings will allow a smaller device, as might be implied if the support device in the figures were considered to be drawn to scale.

As shown in FIG. 10, fenestration support structure 132 of the graft delivery system is then radially expanded to assume a substantially spherical shape in order to press unsupported body portion 544 of graft 540 against the vessel wall having branch vessels 868. The expanded fenestration support structure 132 also models or reduces the wrinkles of graft 540. As explained above, fenestration support structure 132 is radially expanded in situ via proximal retraction of inner shaft 122 relative to intermediate shaft 114.

The expanded fenestration support structure 132 provides support for unsupported body portion 544 of graft 540 such that a fenestration may be created in situ to perfuse side branch vessels 868. As shown in FIG. 11, a separate puncture device 1170 is delivered to create a fenestration in the side wall of graft 540 for perfusion of side branch vessel 868. Puncture device 1170 is delivered through the side branch vessel 868 in a retrograde fashion such that puncture device 1170 is delivered from an opposing side and initially encounters the outside surface of graft 540. Puncture device 1170 may be a dilator-needle combination device having a pointed tip sufficient for puncturing through the material of graft 540. Embodiments of the present structure may be used with any conventional puncture device capable of creating a fenestration in graft 540. Thus, it will be apparent to those of ordinary skill in the art that any features of the puncture device discussed herein are exemplary in nature. For example, the puncture device may be any puncture device known in the art, e.g., biopsy needle, RF dome electrode, or RF ring electrodes, including but not limited to those shown or described in US patent application of Bruszewski et al. Ser. No. 11/939,106, filed 6 Mar. 2008, incorporated in it entirety by reference herein. Once puncture device 1170 is in place adjacent a receiving area of graft 540 where a fenestration is to be created, puncture device 1170 according to its operation punctures the side wall material of graft 540. Expanded fenestration support structure 132 maintains unsupported body portion 544 of graft 540 in a desired position against the vessel wall while the fenestration is created. In addition, expanded fenestration support structure 132 prevents graft 540 from moving during the puncture process. It has been found that to effectively produce robust fenestrations, the graft cloth to be fenestrated must be supported so that some shearing force can be applied. Without such support, even a small lateral force on the graft material (cloth) will result in only a sideways displacement of the graft material, creating a meager fenestration, if one is created at all. Hydrostatic pressure, from blood pressure alone has been found insufficient to create the opposition force needed to create fenestrations predictably and reliably.

If desired, puncture device 1170 may then moved to a second side branch vessel in need of perfusion, and the process is repeated to create additional fenestrations in the side wall of graft 540. Once fenestrations have been created in graft 540 as desired, puncture device 1170 is removed. Fenestration support structure 132 may then be collapsed to an unexpanded straightened configuration by distally advancing the inner shaft as described above, and the graft delivery system may be retracted and removed from the patient. Graft 540 remains expanded in the vessel against the vessel wall to provide an artificial lumen for the flow of blood.

While various embodiments have been described above, it should be understood that they have been presented by way of illustration and example only, and not limitation. It will be apparent to persons skilled in the relevant art that various changes in form and detail can be made therein without departing from the spirit and scope thereof. It will also be understood that each feature of each embodiment discussed herein, and of each reference cited herein, can be used in combination with the features of any other embodiment. All patents and publications discussed herein are incorporated by reference herein in their entirety.

What is claimed is:

1. A graft delivery system for delivering an endoluminal graft to a treatment site within a main vessel having a branch vessel that facilitates fenestration of the endoluminal graft in situ, the system comprising:
a retractable sheath component;
an intermediate shaft slidably disposed within a lumen of the retractable sheath component;
an inner shaft slidably disposed within a lumen of the intermediate shaft;
an expandable fenestration support structure located in a distal portion of the system surrounding the inner shaft, wherein a proximal end of the fenestration support structure is connected to the intermediate shaft and a distal end of the fenestration support structure is connected to the inner shaft, such that the fenestration support structure expands when the distance between the proximal and distal ends thereof is reduced; and
a self-expanding stent graft having a proximal segment, a distal segment, and an unsupported body portion of graft material extending between the proximal and distal segments, the stent graft including at least one self-expanding stent attached to one of the proximal and distal segments for anchoring the stent graft within the main vessel,
wherein the self-expanding stent graft is held between the retractable sheath component and the fenestration support structure when the system is tracked to the treatment site, such that proximal retraction of the sheath component allows the self-expanding stent to deploy within the main vessel at the treatment site.

2. The system of claim 1, wherein the fenestration support structure is expandable to temporarily support the body portion of the stent graft for fenestration of the stent graft in situ.

3. The system of claim 2, wherein the expanded fenestration support structure reduces the wrinkles of the graft material in the body portion of the graft.

4. The system of claim 2, wherein the fenestration support structure is a braided tubular assembly.

5. The system of claim 4, wherein the braided tubular assembly includes metallic wires.

6. The system of claim 5, wherein the metallic wires are formed from a material selected from the group consisting of a stainless steel alloy and nitinol.

7. The system of claim 4, wherein the braided tubular assembly includes polymeric filaments.

8. The system of claim 2, wherein at least portions of the fenestration support structure is coated with a dielectric material.

9. The system of claim 1, wherein the stent graft includes a self-expanding stent attached to each of the proximal and distal segments.

10. A method of creating a fenestration in a tubular endoluminal graft in situ, the method comprising the steps of:
providing a stent graft delivery system, wherein the stent graft delivery system includes an expandable fenestration support structure at a distal portion of the system;
providing a self-expanding stent graft having a proximal segment, a distal segment, and an unsupported body portion of graft material extending between the proximal and distal segments;
loading the self-expanding stent graft on the stent graft delivery system such that the unsupported body portion of the stent graft surrounds the fenestration support structure;
tracking the stent graft delivery system to a target location within a body lumen;
releasing the self-expanding stent graft from the stent graft delivery system such that the stent graft radially expands into apposition with a vessel wall of the body lumen;
radially expanding the fenestration support structure within the unsupported body portion of the stent graft such that the body portion is supported against the vessel wall;
tracking a puncture device to the stent graft such that the puncture device is adjacent to an ostium of a side branch vessel; and
creating a fenestration in the stent graft to perfuse the side branch vessel while the expanded fenestration support structure holds the body portion of the stent graft against the vessel wall.

11. The method of claim 10, further comprising the steps of:
retracting the puncture device;
radially contracting the fenestration support structure to a delivery configuration; and
retracting the stent graft delivery system with the fenestration support structure in the delivery configuration.

12. The method of claim 10, wherein the step of loading the stent graft on the stent graft delivery system includes constraining the stent graft within a retractable sheath component of the stent graft delivery system, and wherein the step of releasing the stent graft includes proximally retracting the sheath component.

13. The method of claim 12, wherein the stent graft includes at least one self-expanding stent attached to one of the proximal and distal segments of the stent graft for expanding and securing the stent graft within the body lumen when the graft is released from the sheath component.

14. The method of claim 13, wherein the stent graft delivery system includes an intermediate shaft disposed within a lumen of the sheath component and an inner shaft disposed within a lumen of the intermediate shaft, wherein a proximal end of the fenestration support structure is connected to the intermediate shaft and a distal end of the expandable support structure is connected to the inner shaft.

15. The method of claim 14, wherein the step of radially expanding the fenestration support structure includes proximally retracting the inner shaft relative to the intermediate shaft.

16. The method of claim 10, wherein the fenestration support structure is a tubular braided component.

17. The method of claim 10, wherein the step of radially expanding the fenestration support structure reduces wrinkles in the stent graft such that the stent graft is modeled.

18. A graft delivery system for delivering an endoluminal graft to a treatment site within a main vessel having a branch vessel that facilitates fenestration of the endoluminal graft in situ, the system comprising:
a self-expanding stent graft having a proximal segment, a distal segment, and an unsupported body portion of graft material extending between the proximal and distal segments, the stent graft including at least one self-expanding stent attached to one of the proximal and distal segments for anchoring the stent graft within the main vessel;
a retractable sheath component; and
a fenestration support structure located in a distal portion of the system, the fenestration support structure being expandable to temporarily support the body portion of the stent graft for fenestration of the stent graft in situ,
wherein the self-expanding stent graft is held between the retractable sheath component and the fenestration support structure when the system is tracked to the treatment site, such that proximal retraction of the sheath component allows the self-expanding stent to deploy within the main vessel at the treatment site.

19. The system of claim 18, wherein the expanded fenestration support structure reduces the wrinkles of the graft material in the body portion of the graft.

20. The system of claim 18, wherein the stent graft includes a self-expanding stent attached to each of the proximal and distal segments.
